(19) United States
(12) Patent Application Publication
(10) Pub. No.: US 2004/0009524 A1
(43) Pub. Date: Jan. 15, 2004

(54) Method for testing hormonal effects of substances

(76) Inventors: Siegmund Wolf, Bad Klosterlausnitz (DE); Maik Obendorf, Weimar (DE); Rene Meyer, Reichenbach (DE); Jens Schroeder, Jena (DE)

Correspondence Address: STRIKER, STRIKER & STENBY, 103 East Neck Road, Huntington, NY, 11743, US

(21) Appl. No.: 10/456,214

(22) Filed: Jun. 6, 2003

(30) Foreign Application Priority Data
Jun. 12, 2002 (DE) 102 26 674.3

(51) Int. Cl. C12Q 1/68

(52) U.S. Cl. 435/6

(57) ABSTRACT

The method for testing of substances for hormonal effects, especially for androgenic or anti-androgenic effects, includes exposing cells transfected with two vectors to the substances, wherein one vector contains a DNA, which codes for a nuclear receptor protein, or a fragment thereof, especially a human nuclear receptor protein, or a fragment thereof, and the other vector contains a DNA, which codes for the FOXG1C co-modulator, or a fragment thereof; and measuring transcription activity, which the nuclear receptor protein, or its fragment, activates or releases in the presence of the FOXG1C co-modulator, or its fragment, and/or measuring the influence of the substance on the interaction between the nuclear receptor protein, or its fragment, and the FOXG1C co-modulator, or its fragment, by protein-protein interaction or protein-protein-DNA interaction. Further a method for determining interference in the co-modulation mechanism between androgen receptor protein and FOXG1C co-modulator is described.

### BACKGROUND OF THE INVENTION

The present invention relates to a method of testing the hormonal effect of substances, and to a method of determining or measuring interference or disturbance in the co-modulation mechanism between androgen receptor proteins and the coactivator Forkhead Box G1C (FOXG1C).

During evaluation of substances for possible pharmaceutical use it is generally common to test these substances for contingent hormonal action, especially for possible androgenic or anti-androgenic activity. Knowledge of the hormonal effects, especially of androgenic or anti-androgenic effects, of these substances is important in many cases in administration of pharmacologically active substances, since they can bring about undesirable side effects in patients. To test the hormonal action of the various substances, the ability of the substances to bind to hormonal receptors and activate their transcription activity is especially measured.

Knowledge of the hormonal effects of substances is of interest not only for potential pharmaceutical, but also for non-pharmaceutical, substances, since it is assumed that many substances present in the surroundings can have androgenic or anti-androgenic and/or estrogenic or anti-estrogenic activity. It is possible that some of these substances produce undesirable deleterious effects.

There is also a considerable need for a method and for a suitable means for performing the method, with which an answer regarding the hormonal effects of substances can be obtained in a reliable, sensitive, simple, economical and rapid manner. The currently known methods are not sufficient.

### SUMMARY OF THE INVENTION

It is therefore an object of the present invention to provide a method and a suitable means for obtaining information regarding hormonal effects of substances to be tested in a reliable, sensitive, simple, economical and rapid manner.

According to the invention this object is attained in a surprising manner by a method for testing of a substance for hormonal effects, especially for androgenic or anti-androgenic effects, comprising the steps of:

a) exposing cells transfected with two vectors to the substance, wherein one vector contains a DNA, which codes for a nuclear receptor protein or a fragment thereof, especially a human nuclear receptor protein or a fragment thereof, and the other vector contains a DNA, which codes for the FOXG1C co-modulator or a fragment thereof; and

b) measuring transcription activity, which the nuclear receptor protein, or its fragment, activates or releases in the presence of the said co-modulator, or its fragment, and/or the influence of the substance on the interaction between the nuclear receptor protein, or its fragment, and said co-modulator, or its fragment, by protein-protein interaction or protein-protein-DNA interaction.

It was surprisingly found that whether or not substances, which, for example, can be environmentally relevant or of pharmacological interest, have a hormonal effect, especially an androgenic or anti-androgenic effect, can be determined with the method according to the invention in a reliable, sensitive, rapid, simple and economical manner.

In the method according to the invention cells transformed with a vector are used. The vector has DNA, which codes for a nuclear receptor protein or a fragment thereof.

The super-family of the nuclear receptors (NRs), to which more than 50 different proteins belong, is a group of related transcription factors, which control the transcription of respective target genes like reactions at specific ligands, e.g. hormones. The families can be divided into subfamilies according to certain characteristics, such as e.g. dimerization status, the type of ligands or the structure of the DNA reaction elements (Beato, et al, Human Reproduction. Update, 6, pp. 225 to 236 (2000)). The conforming or corresponding structure of the functional domain (designated A to F) is a characteristic feature of the NRs. It has a strongly variable, only weakly preservative, N-terminal region with autonomic constitutive activating function (AF-1) a strongly conservative DNA binding domain (DBD), which is responsible for detection of special DNA reaction elements and comprises two zinc finger motifs. It has a variable hinged domain and conservative multifunctional C-terminal ligand-binding domain (LDB) with dimerization and ligand-dependent transactivation function (AF-2). Following that there is a region at the furthest terminal carbon, whose function is not known and absent in some receptors, such as PR (progesterone receptor), PPAR (peroxisome proliferator-activated receptor) and RXR (retinoid-X-receptor) (Mangelsdorf & Evans, Cell, 83, pp. 841-850 (1995); Robyr, et al, Mol. Endocrinol., 14, pp. 329 to 347 (2000)). For a few NRs (e.g. Androgen-receptors (AR)) it is known that the N-terminal region is in a position to interact with the C-terminal region (Brinkmann, et al, J. Steroid Biochem. And Mol. Biol., 69, pp. 307-313 (1999)). Steroid hormone receptors, such as e.g. estrogen (ER), progesterone (PR), glucocorticoid (GR), mineral corticoid (MR) and androgen receptors (AR) bind steroidal ligands, which are derived from pregnenolone, such as progestin, the estrogens, the glucocorticoids and the mineral corticoids, as well as the androgens, bind steroid ligands. The ligand binding activates the receptors and controls expression of the suitable target genes.

As previously explained in step a) of the method according to the invention cells are used which contain a vector which contains DNA coding for the co-modulator FOXG1C or a fragment thereof.

The so-called co-modulators are classes of proteins, which act as bridging molecules between the transcription initiation complex and the NRs in activation (co-activation) and/or repression (co-repression) of gene transcription (McKenna, et al, Endocr. Rev., 20, pp. 321 to 347 (1999)). A co-activator must be able to amplify or magnify the receptor function and to directly integrate with the activated domains of NRs in the presence of an agonist. It must also interact with the basal transcription apparatus and finally it may not amplify the basal transcription activation by itself. Most co-modulators interact with the help of one or more LXXLL-motif(s) (NR boxes) with the AF-2 domain of NRs. However some co-modulators were described which interact with other NR regions (Ding, et al, Mol. Endocrinol., 12, pp. 302 to 313 (1998)). Furthermore many co-modulators were identified, which interact in a similar manner with different NRs, so that the specificity degreee of each co-modulator should be well tested.

In the method according to the invention the co-modulator designated with FOXG1C, or especially the fragment of FOXG1C comprising amino acids 175 to 489, is used. The c-DNA sequence has been described (Genbank XM_{—}007233) and codes for 489 amino acids (Scott, et al, Genomics, 48, pp. 330 to 349 (1998)). A DH5 alpha E. coli clone, which contains plasmids coding for the amino acids 175 to 489 of FOXG1C, was deposited in the German Collection for Microorganisms and Cell Cultures on Jun. 5, 2002 under DSM 15043.

The method according to the invention can be performed using these proteins in an especially reliable, sensitive, simple, economical and rapid manner. Furthermore the FOXG1C fragments, especially the fragment having amino acids 175 to 489 of FOXG1C, have the advantage that they are easily manipulated and cloned, however they still have the functional properties of FOXG1C.

FOXG1C is a co-activator for human androgen receptors and other nuclear receptors, which amplifies the interaction between an androgen and the receptor. The sequence of FOXG1C is already described in Genbank XM 007233; generally no interaction with nuclear receptors, especially the androgen receptor, is described there. The invention is based on the surprising knowledge or understanding that nuclear receptors, especially the AR, on the one hand, and FOXG1C, on the other hand, interact and the AR-mediated transactivation is magnified or augmented. FOXG1C is a protein, which functions as co-mediator, since it amplifies or represses the transcription effect after binding of steroids to the nuclear receptors and promotes binding and activation of nuclear receptors to molecules, to which no hormonal effects were attributed formerly.

FOXG1C represents a co-activator for the androgen receptor and other nuclear receptors, such as estrogen receptor α, estrogen receptor β, progesterone receptor A, progesterone receptor B, glucocorticoid receptor, mineral corticoid receptor, thyroid gland hormone receptor, Vitamin-D receptor, peroxisome proliferator-activer receptor, retinic acid receptor, retinoid X receptor and orphan receptors. These receptors are preferred for use in the present invention, since the advantages of the method according to the invention are obtained in an especially good manner with them.

Vectors, which code for fragments of the preceding or above-mentioned proteins, can also be used in the method according to the invention. The expression “fragments” should be understood in connection with aforementioned proteins, which have an amino acid or several amino acids less than the full-length proteins and still have the functioning properties of a nuclear receptor or a co-modulator.

As already described above, in step a) of the method according to the present invention cells that are transfected with two vectors, which contain DNA coding for special proteins, are used. These cells are thus in a position to express both different proteins.

Preferably the cells are established cell lines and/or eukaryotic cells, especially prostate cells, nerve cells, glia cells, fibroblasts, blood cells, osteoblasts, osteoclasts, hepatocytes, epithelial cells or muscle cells. The method according to the invention can be performed rapidly and economically with the established cell lines. Especially advantageous results can be obtained using the eukaryotic cells, especially the above-described eukaryotic cells.

In a preferred embodiment of the method according to the invention eukaryotic expression vectors are used, e.g. pCMX or pSG5. The method according to the invention can be performed in an especially advantageous and rapid manner and especially outstanding results can be obtained using these vectors, especially in combination with the above-mentioned stable cell lines and/or eukaryotic cells.

Methods for insertion of the DNA coding the preceding proteins in vectors, for introducing the vectors into cells and for culturing the cells so obtained under suitable culture conditions so that these proteins can be expressed, and materials required for those purposes, are known to those skilled in the art.

According to step b) of the method according to the invention the transcription activity is measured, which the nuclear receptor protein or its fragment produces in the presence of the co-modulator or its fragment. This can occur, for example, by detection of a reporter gene.

Reporter genes are genes or gene fragments, which are coupled with other genes or regulator sequences, in order to make the activity of these sequences detectable. Reporter genes produce gene products, which are as easily detectable as possible, for example photometrically by color reaction. Frequently used reporter genes are the genes for β-galactosidase, the gene for alkaline phosphatase, the gene for chloramphenicol-acetyl transferase, the gene for catechol dioxygenase, the gene for the “green fluorescent protein” and different luciferase genes, which induce the cells to produce light.

These reporter genes can likewise be introduced into the cells with vectors, especially eukaryotic expression vectors. For example a vector, which contains DNA coding for a reporter gene, is the MMTV Luciferase vector, which is used for measuring the androgenic activity of substances.

Substances with a hormonal effect, especially with an androgenic/anti-androgenic effect, are then detectable by an elevated or reduced activity of the reporter gene.

The measurement of the influence of the test substance on the interaction between the receptor or its fragment and the co-modulator or its fragment can also occur by determination of the protein-protein interaction. For example, this can take place by twin hybrid systems, immune precipitation, GST pull-down assays, FRET analysis and ABCD assays and determination of protein-protein DNA interaction, for example by gel retardation assays.

It has been found further that FOXG1C can be used as a very good indicator of androgenic-conditioned maladies or illnesses. Relevant androgenic-conditioning illnesses or maladies, such as prostate cancer, erectile dysfunction, infertility, grain or glaze formation, acne or hypogonadism and androgen resistant syndromes, such as testicular feminization, are based on defects in or interference with the co-modulation mechanism between AR and FOXG1C. In patients with these types of illnesses the possibility exists for measurement of relative concentrations of AR and FOXG1C outside the body. This is possible by use of quantitative methods for measuring relative amounts of both molecules in respective patients, in which for example antibodies can be used both against AR and also against FOXG1C or nucleic acid probes can be used against their mRNA. There are several methods for measuring these comparative processes, which are known to one skilled in the art. One skilled in the art also knows suitable materials and apparatus for use in these methods. These methods include radio immunoassay, ELISA tests, immunodyes, RT-PCR, Western Blot or Northern Blot, DNA chip or protein chip. Furthermore it is possible to construct probes for a PCR assay in the usual manner with the help of the FOXG1C-cDNA. Mutations of the normal DNA sequence are detected in certain patients or transcriptions for Northern Blot Assay and/or a DNA for In situ hybridization assays may be produced with these latter probes.

The measured ratio of AR to FOXG1C can be greater or smaller than that required in healthy individuals. The normal value in healthy individuals can be determined in a simple manner, for example by measuring the ratio of AR to FOXG1C in number of healthy test subjects. By comparison of this normal value with that measured in a patient to be tested it can be established whether or not the value in the patient is greater or less than the normal value.

The concentration of FOXG1C and/or AR can be different in different tissues. For example the concentration of FOXG1C in the brain and the testicles is very large, while in contrast its concentration in the liver, heart, thymus and prostate can be comparatively smaller. The different concentrations in the different tissues must be considered during the testing. That means that the test value and the normal or standard value compared should be from the same type of tissue.

Another possibility for determination of defects in the co-modulation mechanism between AR and FOXG1C can be based on only measuring the concentration of FOXG1C, while assuming that the AR concentration is at least approximately constant. If a less than normal FOXG1C concentration is measured, that means that the ratio of AR to FOXG1C has shifted, which suggests interference with the co-modulation mechanism.

It is also possible to determine changes in the FOXG1C expression and thus in the ratio of it to AR with an FOXG1C specific probe. These changes can be involved in starting different illnesses or as consequence of them.

This surprising knowledge that, for example, an androgen resistant syndrome can be based on interference or disturbance of the equilibrium between AR and FOXG1C prevalent in the target cells rests on the finding and characterization of FOXG1C as co-modulator obtained from the measurements of the AR/FOXG1C ratios. Too much FOXG1C can lead to an over-sensitivity of the AR system, so that it reacts to molecules, which normally have no androgenic effect. The reverse leads to a lack of functioning of FOXG1C at all levels of androgen resistance. The detection of too much FOXG1C in patients calls for use of means for down-regulation, such as anti-sense or similar medicines, in order to reduce the FOXG1C titer in certain patients under clinical conditions. This can be achieved by molecules, which are in a position to inhibit the interaction between AR and FOXG1C. If a patient has too little FOXG1C, FOXG1C-cDNA, -protein or -DNA can be administered to him by different known mechanisms, in order to increase the titer of the active FOXG1C in this way. It is possible also to increase the concentration or the activity of the FOXG1C by small molecule drugs or by stimulation of the self-synthesis with the aid of specific FOXG1C promoter proteins.

### BRIEF DESCRIPTION OF THE SEVERAL VIEWS OF THE DRAWING

The objects, features and advantages of the invention will now be illustrated in more detail with the aid of the following description of the preferred embodiments, with reference to the accompanying figures in which:

FIG. 1 is a schematic diagram of the androgen receptor showing the androgen receptor domain (AR 2) from amino acid 325 to 919, which is able to interact with FOXG1C in the presence of androgens;

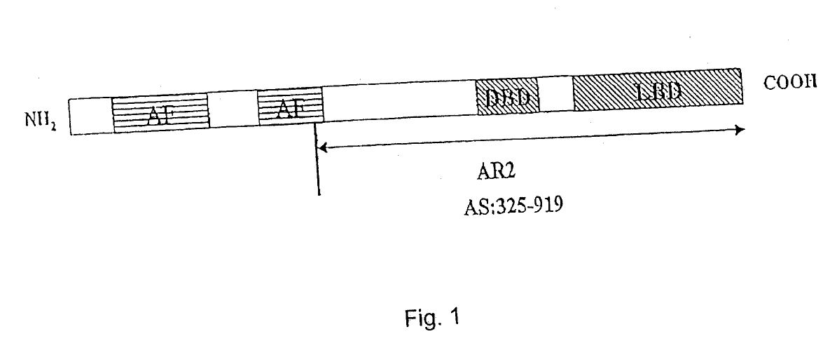

FIG. 2a is an illustration showing the distribution of FOXG1C in various different tissues (human and rodents);

FIG. 2b is an illustration showing the distribution of FOXG1C in different human brain regions;

FIG. 2c is an illustration showing the distribution of FOXG1C in rat brains of young and old animals; and

FIG. 3 is a graphical illustration showing the interaction of FOXG1C or SRC-1a with the androgen receptor in PC3-ARwt cells.

The following examples serve to illustrate the claimed invention without further limiting it.

### EXAMPLES

### Example 1

A screening by a conventional two hybrid yeast system in the presence of androgen 10⁻⁶ M DHT is performed using a cDNA library from fetal brain (Clontech MATCHMAKER) and a human AR fragment, which codes for the amino acids 325 to 919, as probe. In agreement with the recommendations of the manufacturer (Clontech) the number of screened clones amounted to 2×10⁷. The number of independent clones amounts to 3.5×10⁶ according to the public statements of the manufacturer. From those 300 positive clones were selected and tested with a β-galactosidase assay. Of those latter clones 40 were reported as lacZ-positive clones. The inserts of these clones were amplified with PCR. At least 39 different clones were identified by restriction fragment analysis and sequencing. One of those was a clone with an insert comprising 1553 bp (739 bp to 2292 bp), which coded for a part of the ORF (open reading frame) of FOXG1C (Genbank access number XM_{—}007233).

The fragment comprising 1255 bp (963 bp to 2218 bp) of FOXG1C -cDNA sequence served as probe for human Northern Blots. A transcript (3.2 kb) was found in different tissues. The human Northern Blots were hybridized if necessary with a β-Aktin probe, in order to confirm the standard charge on the gel.

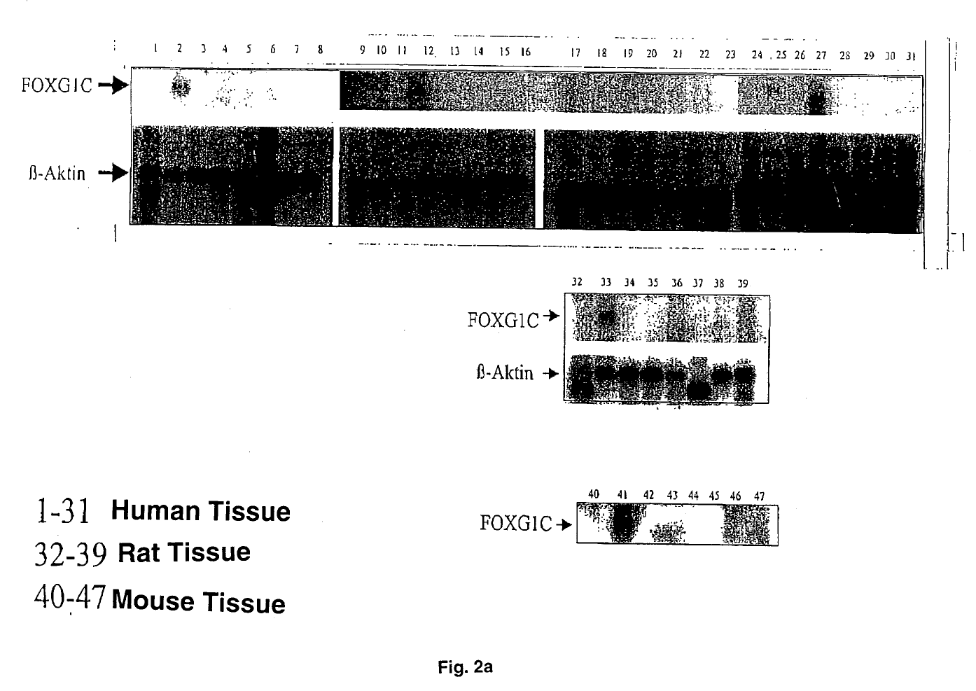

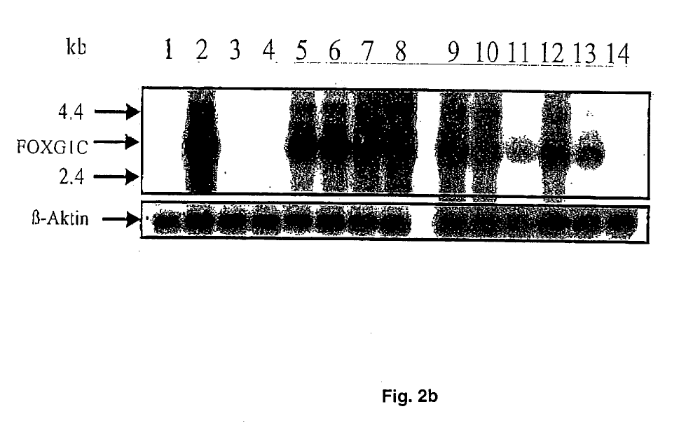

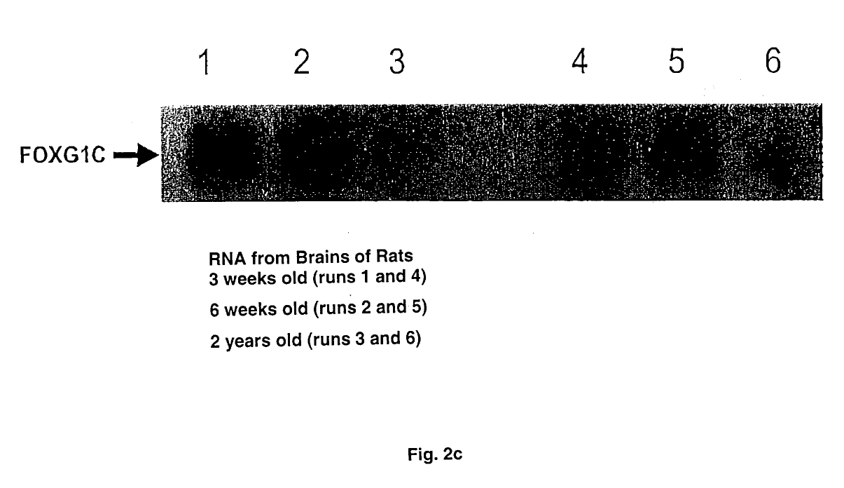

FIG. 2a, 2b and 2c show tissue distributions of FOXG1C, which were measured by a Northern Blot Analysis in the conventional manner. Poly-A⁺-RNA (2 μg) isolated from different human tissues was separated with a formaldehyde-containing agarose gel, blotted on a NYLON® membrane and hybridized with a marked FOXG1C-cDNA fragment (963 to 2218 bp). After washing the membrane was deposited on a film and developed after illumination. As FIG. 2a shows, a very strong expression of FOXG1C was detected in human brain and testicle tissue, while in rodents (rats and mice) the expression of FOXG1C is limited to a large extent in the brain. Furthermore the expression of FOXG1C in human brain sections is reduced or amplified (FIG. 2b). In FIG. 2c 10 μg RNA (run 1-3) or 0.2 μg PolyA⁺ RNA (run 4 to 6) from rat brains were separated on a gel, transferred to a membrane and hybridized with a marked FOXG1C-cDNA fragment (963-2218 bp). The rat brain samples originated from three week old rats (runs 1 and 4), 6 week old rats (run 2 and 5) and 2 year old animals (run 3 and 6). Thus the age dependence of the FOXG1C expression was determined.

The FOXG1C-cDNA fragment from 739 bp to 2218 bp (AS 175-489) from the yeast vector was cloned with the endonucleases EcoRI and Xbal in the usual way in the vector CMX-Vp16 and with MMTV Luciferase in PC3-ARwt cells, which express AR, similarly transfixed.

The distribution of FOXG1C in human tissues is shown by runs 1 to 31, in rat tissue in runs 32 to 39 and in mouse tissue in runs 40 to 47 in FIG. 2a. The human tissues in runs 1 to 31 are taken from the heart (run 1), the brain (run2), the placenta (run 3), the lung (run 4), the liver (run 5), skeletal muscles (run 6), the kidney (run 7), the pancreas (run 8), the kidney (run 9), the thymus (run 10), the prostate (run 11), the testicles (run 12), the ovaries (run 13), the small intestine (run 14), the large intestine (run 15), the peripheral leukocytes (run 16), the stomach (run 17), the thyroid (run 18), the spinal cord (run 19), the lymph nodes (run 20), the trachea (run 21), the adrenal gland (run 21), the spinal cord (run 23), the kidney (run 24), the thymus (run 25), the prostate (run 26), the testicles (run 27), the uterus (run 28), the small intestine (run 29), the large intestine (run 30) and the peripheral leukocytes (run 31). The further tissues, in which the distribution of FOXG1C is measured in FIG. 2a, are the heart (run 32), the brain (run 33), the kidney (run 24), the lung (run 35), the liver (run 36), the skeletal muscles (run 37), the kidney (run 38), the testicles (run 39), the heart (run 40), the brain (run 41), the lung (run 43), the liver (run 44), the skeletal muscles (run 45), the kidney (run 46) and the testicles (run 47).

The distribution of FOXG1C in tissues is shown by runs 1 to 14 in FIG. 2b. This figure shows this distribution in tissues taken from pituitary gland (run 1), cerebral cortex (run 2), the medulla (run 3), the spinal cord (run 4), the rear lob (run 5), the frontal lob (run 6), the temporal lob (run 7), the putamen [sic] (run 8), the tonsils (run 9), the Nucelus caudatus (run 10), the hirnbalken [sic] (run 11), the hippocampus (run 12), the entire brain (run 13) and the thalamus (run 14).

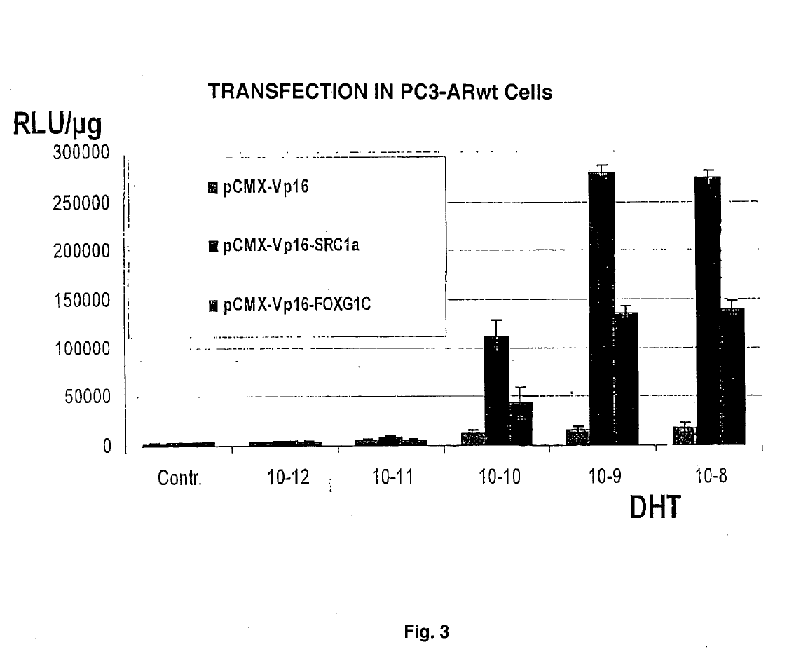

As shown in FIG. 3 the transient transfection of FOXG1C-cDNA fused in frame with the transactivation domain of Vp16 in PC3-ARwt cells leads to a strong co-activation of the AR signal activity. In addition 3×10⁵ cells per cavity in a cell culture dish with cavities were transfixed with 1.0 μg MMTV Luciferase plasmids, with 0.43 μg of the above-described construct in CMX-Vp16 and/or with 0.32 μg CMX-Vp16 (negative control) or 0.5 μg SRC1a-Vp16 (positive control) as control plasmid. The transfixed cells were treated 24 hours with dihydroxytestosterone (DHT) as androgen in the stated concentrations and harvested after another 24 hours, before measuring the activity of the reporter gene (Luciferase). Additionally the entire cell protein amounts were determined for normalization. Two experiments with three measurements each were performed for each transfection initiation and substance concentration. The error variation was reported as SD. The activity is given in relative units.

The disclosure in German Patent Application 102 26 674.3 of Jun. 12, 2002 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a method for testing for hormonal effects of substances, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A method for testing a substance for hormonal effects, said method comprising the steps of:
a) exposing cells transfected with two vectors to the substance, wherein one of the two vectors contains a DNA, which codes for a nuclear receptor protein, or a fragment of said nuclear receptor protein, and another of the two vectors contains a DNA, which codes for a FOXG1C co-modulator, or a fragment of said FOXG1C co-modulator; and
b) measuring transcription activity, which said nuclear receptor protein, or said fragment of said nuclear receptor protein, activates or releases in the presence of said FOXG1C co-modulator or said fragment of said FOXG1C co-modulator, and/or measuring an effect or influence of the substance on an interaction between said nuclear receptor protein or said fragment of said nuclear receptor protein and said FOXG1C co-modulator or said fragment of said FOXG1C co-modulator by protein-protein interaction or by protein-protein-DNA interaction.

2. The method as defined in claim 1, wherein said hormonal effects are androgenic or anti-androgenic effects.

3. The method as defined in claim 1, wherein said nuclear receptor protein is a human nuclear receptor protein or said fragment of said nuclear receptor protein is a fragment of said human nuclear receptor protein.

4. The method as defined in claim 1, wherein said fragment of said FOXG1C co-modulator has amine acids 175 to 489.

5. The method as defined in claim 1, wherein said nuclear receptor protein is selected from the group consisting of androgen receptor, estrogen receptor α, estrogen receptor β, progesterone receptor A, progesterone receptor B, glucocorticoid receptor, mineral corticoid receptor, thyroid gland hormone receptor, Vitamin-D receptor, peroxisome proliferator-activer receptor, retinic acid receptor, retinoid X receptor and orphan receptors.

6. The method as defined in claim 1, wherein said cells are selected from established cell lines and/or are eukaryotic cells.

7. The method as defined in claim 6, wherein said eukaryotic cells are selected from the group consisting of prostate cells, nerve cells, glia cells, fibroblast cells, blood cells, osteoblast cells, osteoclast cells, hepatocytes, epithelial cells and muscle cells.

8. The method as defined in claim 1, wherein said vector is a eukaryotic expression vector.

9. A method for determining interference in a co-modulation mechanism between androgen receptor protein and FOXG1C co-modulator, said method comprising measuring a concentration of FOXG1C co-modulator, or a fragment thereof, and/or a concentration of androgen receptor protein, or a fragment thereof.

10. The method as defined in claim 9, wherein said measuring of said concentration and/or concentrations takes place by radio immunoassay, ELISA test, immunodyeing, RT-PCR, Western Blot or Northern Blot.
